(19) United States
(12) Patent Application Publication
(10) Pub. No.: US 2005/0059653 A1
(43) Pub. Date: Mar. 17, 2005

(54) Novel crystalline forms of 11-cyclopropyl-5,11-dihydro-4-methyl-6H-dipyrido [3,2-b: 2',3'-e][1,4] diazepin-6-one (nevirapine)

(75) Inventors: Buchi Reddy Reguri, Hyderabad (IN); Ramesh Chakka, Hyderabad (IN)

Correspondence Address: LADAS & PARRY, 26 WEST 61ST STREET, NEW YORK, NY, 10023, US

(73) Assignees: DR. REDDY'S LABORATORIES LIMITED; DR. REDDY'S LABORATORIES INC.

(21) Appl. No.: 10/862,892

(22) Filed: Jun. 7, 2004

Related U.S. Application Data
(63) Continuation of application No. 10/601,282, filed on Jun. 20, 2003, now abandoned.

(30) Foreign Application Priority Data
Jun. 21, 2002 (IN) 473/MAS/2002

(51) Int. Cl. A61K 31/551; C07D 487/14

(52) U.S. Cl. 514/220; 540/497

(57) ABSTRACT

The present invention relates to novel crystalline forms of 11-cyclopropyl1-5, 11-dihydro-4-methyl-6H-dipyrido[3,2-b: 2′, 3′-e][1,4]diazepin-6-one, generically known as Nevirapine, and processes for making thereof. More specifically, the present invention provides novel crystalline Form-II and Form-III of Nevirapine.

### BACKGROUND OF THE INVENTION

1. Field of the Invention

The present invention relates to novel crystalline forms of 11-cyclopropyl-5,11-dihydro-4-methyl-6H-dipyrido [3,2-b: 2′,3′-e][1,4] diazepin-6-one, generically known as Nevirapine, and marketed under the trade name of “Viramune”, and processes for making the novel crystalline forms. More specifically, the present invention provides crystalline Form-II and Form-III of Nevirapine.

2. Description of the Prior Art

Nevirapine, which is marketed under the trade name of “Viramune” can be represented by the following Formula (1):

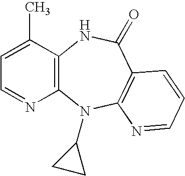

Nevirapine and its pharmaceutically acceptable salts are known and Nevirapine is known as an antiviral drug useful for the treatment of HIV-1 infection in humans comprising a non-nucleoside inhibitor of HIV-1 reverse transcryptase.

The prior art methods do not disclose crystalline polymorphs of Nevirapine.

There is a need to develop pure crystalline polymorphs of Nevirapine that are stable for extended periods of time and suitable for pharmaceutical formulations that exhibit superior bioavailabilty and higher activity compared to the known final product of Nevirapine and processes for making crystalline polymorphs of Nevirapine that are simple, non-hazardous and easily scalable for commercial production.

### SUMMARY OF THE INVENTION

The present invention provides pure crystalline polymorphic forms of Nevirapine that exhibit superior bioavailabilty and higher activity and processes for making crystalline polymorphic forms whereby the processes are simple, non-hazardous and easily scalable for commercial production. The crystalline forms of present invention are pure so the forms satisfy the pharmaceutical requirements and specifications. Furthermore, the pure crystalline forms of the present invention are high melting solids, very suitable for formulation.

More specifically, the present invention provides novel crystalline forms of Nevirapine designated as Form-II and Form-III for convenience and processes for preparing different crystalline forms of Nevirapine from different solvents.

The novel crystalline forms of Nevirapine are also useful as anti-psychotic agents.

The crystalline Form-I of Nevirapine is disclosed in co-pending Indian Patent Application No. 293/MAS/2002, dated Apr. 17, 2002 and entitled “An improved process for the preparation of crystalline polymorph Form-I of Nevirapine”. The process comprises the crystallization of Nevirapine in solvents such as alcohols, ketones, ethers and esters or mixtures thereof.

The crystalline nature of the polymorphs were analyzed using x-ray diffraction. The crystalline forms of present invention are thermally stable and free flowing solids. In general, the free flowing solids are recommended for pharmaceutical formulations and, therefore, the crystalline Form-II and Form-III of Nevirapine are well suited for pharmaceutical applications.

The processes for preparing different crystalline forms of Nevirapine of the present invention are simple, non-hazardous and easily scalable for commercial production. The process for the preparation of crystalline Form-II of Nevirapine comprises the recrystallization of Nevirapine in solvents such as aromatic hydrocarbons or alcohols or ketones. The process for the preparation of crystalline Form-III of Nevirapine comprises the recrystallistion of Nevirapine in halogenated solvents. The processes of the present invention are commercially viable and well suited for industrial scale up.

Other objects, advantages and features of the present invention will become apparent to those skilled in the art from the following discussion.

### BRIEF DESCRIPTION OF THE ACCOMPANYING DRAWINGS

FIG. 1 is an X-ray powder diffractogram of a sample of the crystalline Form-II of Nevirapine.

FIG. 2 is an X-ray powder diffractogram of a sample of the crystalline Form-III of Nevirapine.

### DETAILED DESCRIPTION OF THE INVENTION

The present invention arose from the desire by the inventors to improve the bioavailaibility and activity of Nevirapine through the use of different crystalline forms of Nevirapine. After reviewing the literature on Nevirapine and their methods of preparation, the inventors concluded that it was desirable to provide novel crystalline forms of Nevirapine and a process for preparing the novel crystalline forms of Nevirapine that exhibit superior bioavailabilty and higher activity and will, thus, afford an improved therapeutic profile.

Accordingly, this patent provides novel crystalline forms II and III of Nevirapine and processes for preparing the novel crystalline forms II and III of Nevirapine.

The novel crystalline forms of Nevirapine of the present invention are characterized by their X-ray diffractograms, Differential Scanning colorimetry thermograms and IR spectra. The X-ray diffraction patterns of Form-II and Form-III of Nevirapine were measured on a Bruker Axe, DS Advance Powder X-ray Diffractometer with Cu K alpha-1 Radiation source. The 2-theta values and their intensity percentages of relevant peaks in X-ray powder diffraction pattern of crystalline Form-II and Form-III of Nevirapine are shown in the Table-1.

TABLE 1

| | |
|---|---|
| Form-II | Form-III |

| | Intensity | | Intensity |
|---|---|---|---|
| 2 theta (^{o}) | (I/Io) | 2 theta (^{o}) | (I/Io) |
| 9.51 | 100 | 13.072 | 100 |
| 13.287 | 49.1 | 25.509 | 88.1 |
| 25.752 | 39.2 | 13.468 | 63.3 |
| 13.706 | 28.3 | 22.805 | 59.2 |
| 19.258 | 26.8 | 14.077 | 55 |
| 26.904 | 25.7 | 19.027 | 52.4 |
| 22.842 | 19.9 | 9.264 | 51.2 |
| 25.317 | 19.9 | 24.537 | 41.4 |
| 21.03 | 17.2 | 11.202 | 36.6 |
| 23.445 | 15.8 | 21.289 | 35.3 |
| 17.473 | 13.7 | 19.846 | 29.8 |
| 15.636 | 9 | 25.09 | 29 |
| 23.996 | 8.4 | 26.47 | 28 |
| 12.84 | 7.6 | 17.217 | 27.7 |
| 29.063 | 7.4 | 26.663 | 24.3 |
| 20.56 | 7.2 | 23.218 | 22.9 |
| 29.97 | 6.7 | 15.412 | 20.6 |
| 34.176 | 6.5 | 23.688 | 20.5 |
| 16.974 | 6.3 | 20.754 | 19.4 |
| 33.13 | 6.2 | 12.657 | 19.1 |
| 27.432 | 4.4 | 27.674 | 18.1 |
| 27.93 | 3.8 | 27.217 | 17 |
| 28.459 | 3.3 | 24.024 | 15.8 |
| 32.072 | 3.3 | 28.342 | 13.3 |
| 35.139 | 2.2 | 20.376 | 12.8 |
| 31.369 | 1.7 | 29.718 | 12.3 |
| | | 33.904 | 10.8 |
| | | 15.705 | 10.3 |
| | | 16.736 | 8.7 |
| | | 28.824 | 8.2 |
| | | 32.89 | 8.1 |
| | | 37.192 | 7 |
| | | 29.216 | 5.6 |
| | | 38.082 | 4.3 |

Most pharmaceuticals formulation processes are facilitated by the use of active materials that are free flowing high melting solids. The crystalline forms of present invention are high melting solids, very suited for formulation.

Moreover, the novel crystalline polymorphs of the present invention are also stable for extended periods of time without need of specialized storage conditions.

The novel crystalline forms of Nevirapine are useful as anti-psychotics.

The relevant X-ray diffractograms of crystalline Form-II and Form-III of Nevirapine are depicted in FIGS. (1) and (2) respectively.

The crystalline Form-II and Form-III of Nevirapine are also characterized by their Differential Scanning Colorimetry thermograms, which is analyzed on Schimadzu DSC-50 in a temperature range of 25-230° C. with a heating rate of 5° C./minute under Nitrogen with a flow rate of 50.0 ml/minute.

The Differential Scanning Colorimetry thermogram of Form-II and Form-III exhibits a significant endo peaks around 247° C. and 246° C. respectively.

The crystalline Form-II and Form-III of present invention are further characterized by their Infra red spectral data, which are measured by KBr-transmission method on Perkin-Elmer FT-IR instrument. The identified significant IR bands of these forms are mentioned in the following Table-2.

TABLE 2

| Form-II | Form-III |
|---|---|
| Wavelength | Wave length |
| (Cm⁻¹) | (Cm⁻¹) |
| 461.89 | 462.50 |
| 540.17 | 697.02 |
| 621.10 | 789.09 |
| 696.95 | 829.67 |
| 761.89 | 884.83 |
| 788.95 | 1025.60 |
| 884.52 | 1242.25 |
| 1074.45 | 1289.81 |
| 1242.40 | 1354.63 |
| 1354.59 | 1383.17 |
| 1413.41 | 1414.39 |
| 1586.57 | 1465.21 |
| 1646.14 | 1586.43 |
| 3061.90 | 1647.22 |
| 3188.74 | 3062.51 |
| | 3189.72 |

Another aspect of the present invention is to prepare the novel crystalline forms of Nevirapine.

The process for the preparation of crystalline Form-II of Nevirapine comprises dissolving crude Nevirapine in a solvent selected from aromatic hydrocarbon solvents, alcohol, ketone solvents, or mixtures of any of these solvents to form a reaction solution that is clear in color. The reaction solution may optionally be treated with carbon. The solvent of the reaction solution may optionally be distilled to a minimum volume in a vacuum environment or normal atmosphere from the reaction solution. The reaction solution is subsequently cooled to a temperature of 0-35° C., preferably to 0-10° C., accompanied by stirring until a crude compound of Form-II of Nevirapine crystallizes. The separated solid is filtered to obtain the crystalline Form-II of Nevirapine. The solid may optionally be washed and dried at a temperature of 30-90° C. to afford the desired crystalline Form-II of Nevirapine.

Non-limiting examples of aromatic hydrocarbon solvents include benzene, toluene, ethyl benzene or xylene. A preferred example of an aromatic hydrocarbon is toluene. A non-limiting example of an alcohol includes n-butanol. A non-limiting example of a ketone solvent includes methyl iso butyl ketone.

The process for the preparation of crystalline Form-Ill of Nevirapine comprises dissolving crude Nevirapine in halo solvents selected from chloroform, dichloromethane or dichloroethane, preferably chloroform, at the reflux temperature of the solvent to form a reaction solution. The reaction solution may optionally be treated with carbon. A halo solvent, preferably dichloromethane, is subsequently added to the reaction solution until a crude compound of Form-Ill of Nevirapine crystallizes. The separated solid is filtered to obtain the crystalline Form-III of Nevirapine. The solid may optionally be washed and dried at a temperature of 30-90° C. to afford the desired crystalline Form-III of Nevirapine.

The processes of the present invention are simple, and easily scalable for commercial production.

The Form-II and Form-III of Nevirapine are obtained in pure and crystalline form to enable formulations and to meet the pharmaceutical requirements and specifications.

The present invention will now be illustrated by means of examples that are provided for illustration purposes only, and are not intended to limit the effective scope of either the invention or the claims.

### EXAMPLES

### REFERENCE EXAMPLE

### Preparation of Crude Nevirapine

2-Chloro-N-(2-chloro-4-methyl-3-pyridyl)-3-pyridine carboxamide (150 grams, 0.5319 moles), calcium oxide (30 grams, 0.5357 moles), cyclopropylamine (95.1 grams, 1.6684 moles) in diglyme (300 ml) was heated to a temperature of 135-145° C. until the reaction was completed to form a reaction mass. The reaction mass was subsequently cooled to temperature of 20-30° C., filtered and washed with diglyme (150 ml). Half of the initial volume of the solvent was distilled off from the filtrate under vacuum before diglyme (37.5 ml) was added at a temperature of 50-60° C. This reaction mass was slowly added to a hot suspension of sodium hydride (57.6 grams, 1.44 moles) in diglyme (105 ml) at about 140° C. The reaction mass was maintained at a temperature of 140° C. for about 30-60 minutes. The reaction mass was then cooled to a temperature of 40-50° C. before ethyl acetate (360 ml) was added and further cooled to a temperature of 0-10° C. Acetic acid (103 ml) followed by water (105 ml) was added to the reaction mass and stirred for 1-2 hours, filtered the separated compound and washed with ethyl acetate (60 ml) The compound was dried at a temperature of 60-80° C. to afford the crude Nevirapine.

(Weight: 124.8 grams)

### Example 1

### Preparation of Novel Crystalline Form-II of Nevirapine

A mixture of crude Nevirapine (10.0 grams), as prepared per Reference Example, and toluene (250 ml) were heated to the reflux temperature to obtain a clear solution. Carbon (2.0 grams) was added and stirred for 5 minutes to form a reaction mass. The reaction mass was subsequently filtered and cooled to a temperature of 0-10° C. and stirred for 2-3 hours to crystallize the solid mass. The crystalline solid mass was filtered, washed with toluene (10.0 ml) and dried to obtain the crystalline Form-II of Nevirapine.

(Weight: 7.5 grams)

### Example 2

### Preparation of Novel Crystalline Form-II of Nevirapine

A mixture of crude Nevirapine (5.0 grams), as prepared per Reference Example, and n-butanol (100 ml) were heated to the reflux temperature to obtain a clear solution. Carbon (1.0 grams) was added and stirred for 10-15 minutes to form a reaction mass. The reaction mass was subsequently filtered and cooled to a temperature of 0-10° C. and stirred for 1-2 hours to crystallize the solid mass. The crystalline solid mass was filtered, washed with n-butanol (5.0 ml) and dried to obtain the crystalline Form-II of Nevirapine.

(Weight: 3.1 grams)

### Example 3

### Preparation of Novel Crystalline Form-II of Nevirapine

A mixture of crude Nevirapine (5.0 grams, 0.0187 moles), as prepared per Reference Example, and methyl isobutyl ketone (225 ml) were heated to the reflux temperature to obtain a clear solution. Carbon (1.0 grams) was added and stirred for 10-15 minutes to form a reaction mass. The reaction mass was subsequently filtered and cooled to a temperature of 0-10° C. and stirred for 1-2 hours to crystallize the solid mass. The crystalline solid mass was filtered, washed with methyl iso butyl ketone (5.0 ml) and dried to obtain the crystalline Form-II of Nevirapine.

(Weight: 2.8 grams)

### Example 4

### Preparation of Novel Crystalline Polymorph Form-III of Nevirapine

A mixture of crude Nevirapine (5.0 grams), as prepared per Reference Example, and chloroform (35.0 ml) were heated to the reflux temperature to obtain a clear solution. Carbon (1.0 grams) was added and stirred for 5-10 minutes to form a reaction mass. The reaction mass was subsequently filtered and transferred into a fresh round bottomed flask. Dichloro ethane (75.0 ml) was added slowly to the reaction mixture at a temperature of 25-35° C. to precipitate the compound. The obtained crystalline solid mass was stirred for 30-60 minutes and then filtered and accompanied by drying at a temperature of 50-70° C. to obtain crystalline Form-III of Nevirapine.

(Weight: 3.0 grams)

### DETAILED DESCRIPTION OF THE ACCOMPANYING DRAWINGS

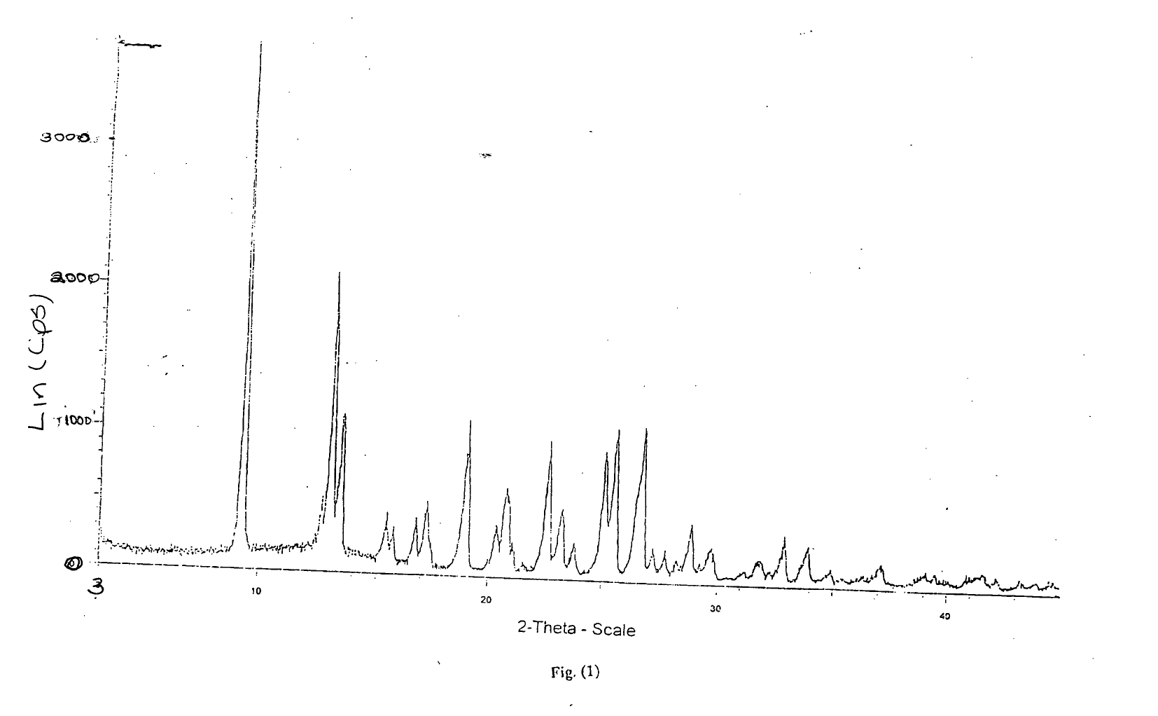

FIG. 1 is the characteristic X-ray powder diffraction pattern of a sample of the crystalline Form-II of Nevirapine.

Vertical axis: Intensity (CPS); Horizontal axis: 2 Theta (degrees).

The significant 2-theta values (in degrees) were obtained around about 9.51, 12.84, 13.287, 13.706, 15.636, 16.974, 17.473, 19.258, 20.56, 21.03, 22.842, 23.445, 23.996, 25.317, 25.752, 26.904, 27.432, 27.93, 28.459, 29.063, 29.97, 31.369, 32.072, 33.13, 34.176 and 35.139 degrees two theta.

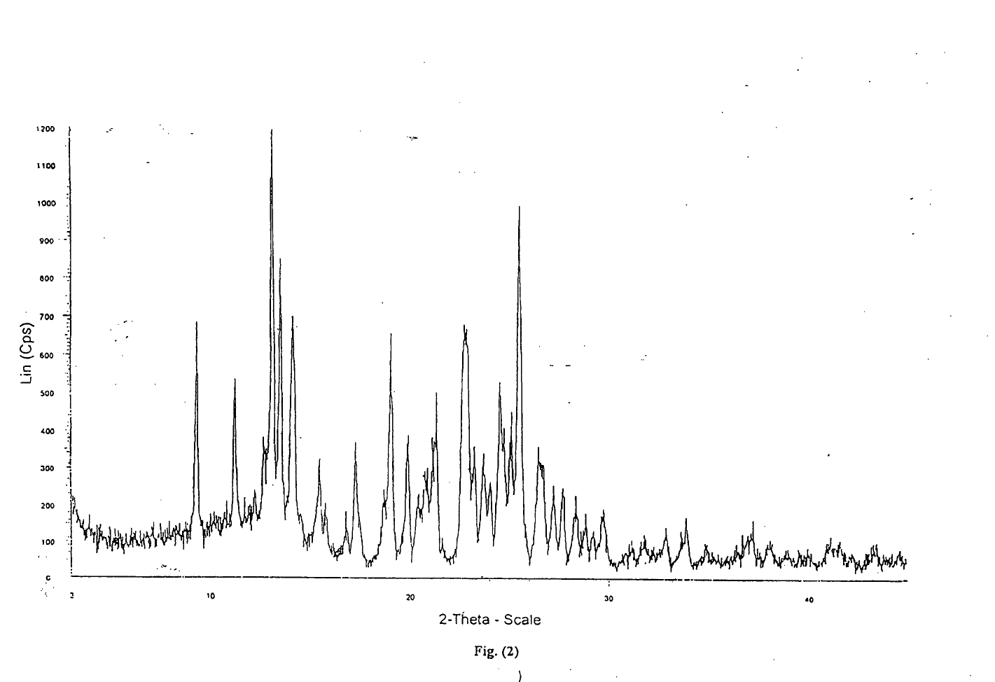

FIG. 2 is the characteristic X-ray powder diffraction pattern of a sample of the crystalline Form-III of Nevirapine.

Vertical axis: Intensity (CPS); Horizontal axis: 2 Theta (degrees).

The significant 2-theta values (in degrees) were obtained around about 9.264, 11.202, 12.657, 13.072, 13.468, 14.077, 15.412, 15.705, 16.736, 17.217, 19.027, 19.846, 20.376, 20.754, 21.289, 22.805, 23.218, 23.688, 24.024, 24.537, 25.09, 25.509, 26.47, 26.663, 27.217, 27.674, 28.342, 28.824, 29.216, 29.718, 32.89, 33.904, 37.192 and 38.082 degrees two theta.

The invention now being fully described, it will be apparent to one of ordinary skill in the art that many changes and modification can be made thereto without departing from the spirit or scope of the invention as set forth herein.

1. Novel crystalline Form-II of Nevirapine.

2. The crystalline Form-II of Nevirapine according to claim 1 comprising the following X-ray powder diffraction pattern: (2-theta values in degrees) of about 9.51, 12.84, 13.287, 13.706, 15.636, 16.974, 17.473, 19.258, 20.56, 21.03, 22.842, 23.445, 23.996, 25.317, 25.752, 26.904, 27.432, 27.93, 28.459, 29.063, 29.97, 31.369, 32.072, 33.13, 34.176 and 35.139 degrees two theta.

3. The crystalline Form-II of Nevirapine according to claim 1 having an X-ray powder diffraction pattern substantially as depicted in FIG. 1.

4. A process for the preparation of novel crystalline Form-II of Nevirapine, which comprises the steps of:
(i) dissolving crude Nevirapine in a solvent selected from aromatic hydrocarbon solvents, alcohol, ketone solvents or mixtures thereof to obtain a reaction solution;
(ii) optionally treating the reaction solution with carbon;
(iii) optionally distilling the solvent from the reaction solution of step (ii) to a minimum volume to form a reaction mass;
(iv) cooling the reaction mass to a temperature of 0-35° C., accompanied by stirring the reaction mass until a solid precipitates;
(v) filtering the solid from step (iv);
(vi) optionally washing the solid obtained in step (v);
(vii) drying the solid of step (vi) at a temperature of 30-90° C. to obtain the crystalline Form-II of Nevirapine.

5. The process according to claim 4 where the aromatic hydrocarbon solvent is selected from benzene, toluene, ethylbenzene or xylene.

6. The process according to claim 5 where the aromatic hydrocarbon is toluene.

7. The process according to claim 4 where the alcohol is n-butanol.

8. The process according to claim 4 where the ketone solvent is methyl iso butyl ketone.

9. The process according to claim 4 wherein the optionally distilling step (iii) occurs in a vacuum.

10. Novel crystalline Form-III of Nevirapine.

11. The crystalline Form-III of Nevirapine according to claim 10 which has the following X-ray powder diffraction pattern: (2-theta value in degrees) of about 9.264, 11.202, 12.657, 13.072, 13.468, 14.077, 15.412, 15.705, 16.736, 17.217, 19.027, 19.846, 20.376, 20.754, 21.289, 22.805, 23.218, 23.688, 24.024, 24.537, 25.09, 25.509, 26.47, 26.663, 27.217, 27.674, 28.342, 28.824, 29.216, 29.718, 32.89, 33.904, 37.192 and 38.082 degrees two theta.

12. The crystalline Form-III of Nevirapine according to claim 10 having an X-ray powder diffraction pattern substantially as depicted in FIG. 2.

13. A process for the preparation of crystalline Form-III of Nevirapine, which comprises the steps of:
(i) dissolving crude Nevirapine in a halo solvent selected from chloroform, dichloromethane or dichloroethane at a reflux temperature of the halo solvent to form a reaction solution;
(ii) optionally treating the reaction solution with carbon;
(iii) adding a second halo solvent to the reaction solution until a solid precipitates;
(iv) filtering the solid from step (iii);
(v) optionally washing the solid from step (iv);
(vi) drying the solid of step (v) at a temperature of 30-90° C. to obtain the crystalline Form-III of Nevirapine.

14. The process according to claim 13 wherein the halo solvent of step (i) is chloroform.

15. The process according to claim 13 wherein the halo solvent of step (iii) is dichloroethane.
